(19) United States
(12) Patent Application Publication
(10) Pub. No.: US 2012/0116221 A1
(43) Pub. Date: May 10, 2012

(54) METHODS AND SYSTEMS FOR IMAGE-GUIDED TREATMENT OF BLOOD VESSELS

(75) Inventors: Chandra Sehgal, Wayne, PA (US); Andrew Kenneth Wood, Philadelphia, PA (US)

(73) Assignee: The Trustees of the University of Pennsylvania, Philadelphia, PA (US)

(21) Appl. No.: 13/263,133

(22) PCT Filed: Apr. 9, 2010
(86) PCT No.: PCT/US10/30519

§ 371 Date: Jan. 20, 2012

Related U.S. Application Data
(60) Provisional application No. 61/168,075, filed on Apr. 9, 2009.

(51) Int. Cl. A61N 7/00; A61B 8/00

(52) U.S. Cl. 600/439

(57) ABSTRACT

Methods and systems of treating at least one blood vessel involves the application of therapy ultrasound to the blood vessel(s) using one or more dosing conditions. An image of the region of interest is acquired responsive to the applied therapy ultrasound. A change in vascularity of the blood vessel(s) is estimated, responsive to the applied therapy ultrasound, using the acquired image to determine whether to adjust at least one of the dosing conditions. The therapy ultrasound is applied with an intensity to modify the blood vessel(s) without damaging a surrounding tissue. A method of treating a tumor comprises introducing a therapeutic agent into a bloodstream and applying therapy ultrasound to blood vessel(s). The therapy ultrasound, along with an agent, disrupts the blood vessel(s) to limit flow to and from the tumor, thereby retaining the therapeutic agent within the tumor.

### CROSS REFERENCE TO RELATED APPLICATIONS

This application is related to and claims the benefit of U.S. Provisional Application No. 61/168,075 entitled METHODS AND SYSTEMS FOR IMAGE-GUIDED TREATMENT OF BLOOD VESSELS filed on Apr. 9, 2009, the contents of which are incorporated herein by reference.

### STATEMENT REGARDING FEDERALLY SPONSORED RESEARCH

This invention was made with partial government support under the grants EB 001713 and CA 139657 awarded by the National Institutes of Health. The government has certain rights in the invention.

### FIELD OF THE INVENTION

The present invention relates to ultrasound imaging and therapy. More particularly, the present invention relates to methods and systems of image-guided treatment of blood vessels with low intensity ultrasound.

### BACKGROUND OF THE INVENTION

It is generally known to use ultrasound for clinical imaging of a region of a patient's anatomy. For clinical imaging, an ultrasound transducer transmits ultrasound waves to a subcutaneous body structure, such as lesions, blood vessels and internal organs. The ultrasound waves are reflected from the target structure and processed to generate an image of the target structure.

It is also generally known to use ultrasound for therapeutic applications, for example, to treat cysts, tumors and kidney stones. For therapeutic applications, the ultrasound waves are typically applied with an energy that is much greater than for clinical imaging. For example, the intensity of imaging ultrasound is typically in the range of about 10-60 mW/cm², whereas the intensity of physiotherapy ultrasound is typically in the range of about 0.5-3 W/cm². Therapeutic ultrasound generally provides regional heating or regional mechanical changes in a target body structure. One type of therapeutic ultrasound includes high intensity focused ultrasound (HIFU), also known as focused ultrasound (FUS), which typically has an intensity of about 1000-10,000 W/cm², and generally produces a highly localized heating of the target body structure.

For therapeutic ultrasound, an imaging transducer may be used to aid in positioning a therapeutic transducer to the treatment area, in order for the therapeutic transducer to suitably administer the therapeutic ultrasound. The imaging transducer may also be used to monitor an extent of the therapeutic response (such as whether a blood clot is dissolved). Typically, imaging and therapeutic ultrasound are performed separately, because simultaneous application may introduce artifacts in the acquired image. Even with use of imaging ultrasound, it is typically difficult to evaluate the extent of the applied therapeutic treatment. It may also be difficult to obtain an objective measure indicating that the therapeutic treatment is complete.

### SUMMARY OF THE INVENTION

The present invention relates to methods and systems of treating at least one blood vessel in a region of interest. Therapy ultrasound is applied to the at least one blood vessel within the region of interest using one or more dosing conditions. An image of the region of interest is acquired responsive to the applied therapy ultrasound. A change in vascularity of the at least one blood vessel is estimated, responsive to the applied therapy ultrasound, using the acquired image to determine whether to adjust at least one of the dosing conditions. The therapy ultrasound is applied with an intensity to modify the at least one blood vessel without damaging a surrounding tissue.

The present invention also relates to a method of disrupting at least one blood vessel in a region of interest. The method includes: a) directing an agent including microbubbles to the at least one blood vessel in the region of interest, b) applying therapy ultrasound to the at least one blood vessel within the region of interest using one or more dosing conditions, the microbubbles interacting with the therapy ultrasound to disrupt the at least one blood vessel, c) acquiring an image of the region of interest responsive to the applied therapy ultrasound, d) estimating a change in vascularity of the at least one blood vessel, responsive to the applied therapy ultrasound, using the acquired image to determine whether to adjust at least one of the dosing conditions and e) repeating steps (b)-(d) until the at least one blood vessel is disrupted in accordance with a predetermined treatment response. The applied therapy ultrasound is applied with an intensity to disrupt the at least one blood vessel without damaging a surrounding tissue.

The present invention further includes a method of treating a tumor with a therapeutic agent. The therapeutic agent is introduced into a bloodstream to be directed to the tumor. An agent including microbubbles is directed to blood vessels associated with the tumor in a region of interest. Therapy ultrasound is applied to the blood vessels within the region of interest such that the microbubbles interact with the therapy ultrasound to disrupt at least one of the blood vessels. The therapy ultrasound is applied with an intensity to disrupt the at least one of the blood vessels without damaging a surrounding tissue to limit flow to and from the tumor and to retain the therapeutic agent within the tumor.

### BRIEF DESCRIPTION OF THE DRAWINGS

The invention may be understood from the following detailed description when read in connection with the accompanying drawings. It is emphasized that, according to common practice, various features/elements of the drawings may not be drawn to scale. On the contrary, the dimensions of the various features/elements may be arbitrarily expanded or reduced for clarity. Moreover, in the drawing, common numerical references are used to represent like features/elements. Included in the drawing are the following figures:

FIG. 1 is a functional block diagram of an exemplary system for treating blood vessels, according to an embodiment of the present invention;

FIGS. 2A and 2B are functional block diagrams of configurations of imaging and therapeutic transducers used with the system shown in FIG. 1, according to embodiments of the present invention;

FIGS. 3A, 3B and 3C are diagrams of a vascular system illustrating the effect of microbubbles on the vascular system when insonated by therapeutic ultrasound using the system shown in FIG. 1;

FIG. 4 is a flow chart illustrating an exemplary method of treating blood vessels, according to an embodiment of the present invention;

FIG. 5A is a flow chart illustrating an exemplary method of determining an initial vascularity, according to an embodiment of the present invention;

FIG. 5B is a flow chart illustrating an exemplary method of estimating a change in vascularity with application of therapeutic ultrasound, according to an embodiment of the present invention;

FIG. 6 is a flow chart illustrating an exemplary method of treating a tumor with a therapeutic agent, according to an embodiment of the present invention;

FIG. 7 is a perspective view of an exemplary system for treating varicose veins, according to an embodiment of the present invention;

FIG. 8 is a perspective view of an exemplary system for treating macular degeneration, according to an embodiment of the present invention;

FIG. 9 is a graph illustrating an example of survival probability as a function of time with application of an antivascular therapy in accordance with an embodiment of the present invention;

FIGS. 10A and 10B are graphs illustrating an example of tumor growth as a function of time for a control group and with application of an antivascular therapy, respectively, in accordance with an embodiment of the present invention;

FIG. 11 is a graph illustrating another example of survival probability as a function of time with application of an antivascular therapy in accordance with an embodiment of the present invention;

FIG. 12 is a graph illustrating an example of normalized density distribution of microbubbles in various contrast agents as a function of radius;

FIG. 13 is a graph illustrating an example of temperature as a function of frequency due to ultrasonic heating in the presence of microbubbles in accordance with an embodiment of the present invention;

FIG. 14 is a graph illustrating an example of temperature as a function of concentration with application of sonication at various frequencies in accordance with an embodiment of the present invention;

FIG. 15 is a graph illustrating an example of microbubble-induced heating as a function of frequency with application of various ultrasound intensities in accordance with an embodiment of the present invention;

FIG. 16 is a graph illustrating an example of temperature as a function of time with application of continuous and intermittent sonication in accordance with an embodiment of the present invention; and

FIG. 17 is a graph illustrating an example of temperature as a function of blood flow rate with application of ultrasound for various fractional interaction times with microbubbles in accordance with an embodiment of the present invention.

### DETAILED DESCRIPTION OF THE INVENTION

Aspects of the present invention include methods and systems of treating at least one blood vessel with therapy ultrasound. The therapy may be guided by acquiring images over a therapy period. The images may be used to estimate a change in vascularity of the blood vessel, responsive to the applied therapy ultrasound. The estimated change in vascularity may be used to adjust or maintain the dosing conditions during treatments performed in the therapy period. According to aspects of the present invention, the therapy ultrasound may be applied with an intensity to modify the at least one blood vessel without damaging a surrounding tissue. This intensity is generally referred to herein as low intensity ultrasound (LIU).

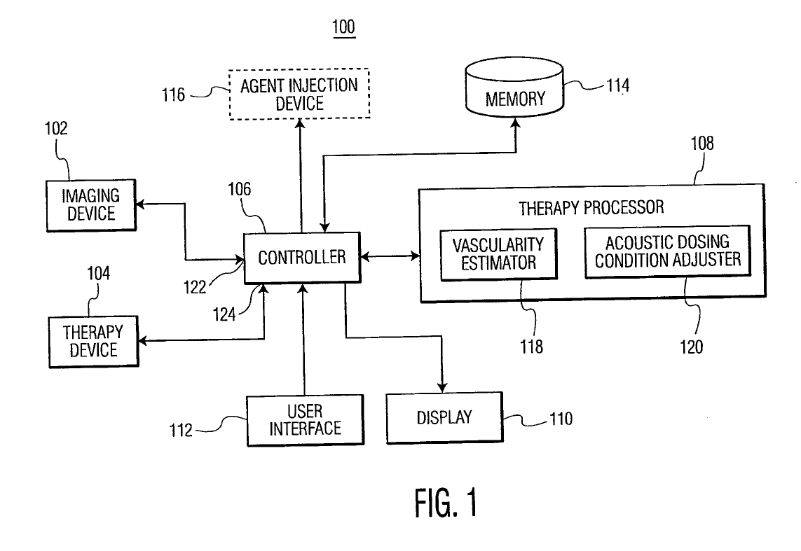

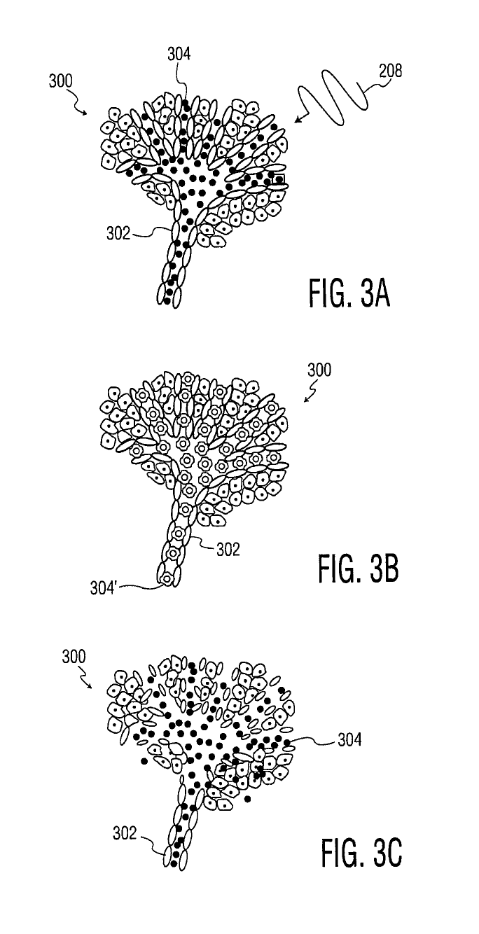

Referring to FIG. 1, an exemplary system 100 for treating blood vessels is shown. System 100 includes imaging device 102, therapy device 104, controller 106, therapy processor 108, display 110, user interface 112 and memory 114. System 100 may also include agent injection device 116, described further below with respect to FIG. 3. Suitable displays 110 and user interfaces 112 will be understood by one of skill in the art from the description herein.

Imaging device 102 is configured to acquire images of a subcutaneous region of a subject over a therapy period, responsive to controller 106. The therapy may be performed over one or more treatment periods during which a treatment is applied to a target blood vessel. As used herein, the target blood vessel may include at least one blood vessel within a region of interest (ROI) of the acquired image. Each treatment period may be separated by a predetermined cessation period. In particular, imaging device 102 may provide images between treatment periods (e.g., prior to treatment and during cessation periods).

In general, imaging device 102 exposes the subcutaneous region to energy waves and measures differences in absorption of the transmitted energy, an energy scattered by the subcutaneous region or an energy released in the presence of the applied energy. It is understood that imaging device 102 may include any suitable device for acquiring an image of a body structure, for example, magnetic resonance imaging (MRI), computerized tomography (CT) scanning, positron emission tomography (PET) scanning, radionuclide scanning, X-ray and ultrasound imaging.

Therapy device 104 is configured to apply therapy ultrasound to the target blood vessel within the ROI, responsive to controller 106. According to an exemplary embodiment, therapy device 104 is configured to provide low intensity ultrasound (LIU) with an intensity of less than about 5.0 W/cm². According to another exemplary embodiment, the intensity of therapy device 104 is between about 0.01 W/cm² and about 5 W/cm² (defined with respect to spatial average temporal average (ISATA)). A preferred range of intensities is about 2 W/cm² or less. By applying LIU, therapy device 104 applies an intensity that is sufficient to modify the targeted blood vessel, without damaging the surrounding tissue. According to an exemplary embodiment, the LIU may be applied as continuous waves (i.e. for the duration of the treatment period). It is contemplated that the LIU may also be applied as long tone bursts with different pulse repetition frequencies over the treatment period.

Therapy device 104 may also be configured to apply the LIU with minimal focusing. For example, according to exemplary embodiments, the LIU may be unfocused or mildly focused with a degree of focusing (κ) of less than about 6. The degree of focusing is shown in eq. 1 as:

$$κ = \frac{{r}^{2} / λ}{F} \qquad ( 1 )$$

where λ represents a wavelength of the ultrasound wave, r represents a radius of a transducer of therapy device 104 and F represents a focal length of the transducer of therapy device 104.

For example, system 100, using LIU, may be used to target leaky and fragile preexisting channels and those formed as a result of tumor angiogenesis, without disturbing the healthy blood vessels. Disruption of the tumor-associated blood vessels may be used to treat the tumor itself, by preventing blood flow to the tumor. Accordingly, the present invention may be applicable to various cancers affecting internal and external areas of the body, for example, skin, liver, kidneys, prostate, uterus, breast, etc.

As cancer grows, the upregulation of angiogenic factors results in the sprouting of new blood vessels from pre-existing vessels to supply the cancer with nutrients and oxygen. However, these new vessels fail to mature into a normally functioning vasculature. The vessels tend to be fragile and leaky. The endothelial cells of the vessels remain loosely associated. There is continued degradation of the extracellular matrix, and the basement membrane is discontinuous or may fail to form. The resulting vasculature is not fully functional, has a non-uniform distribution, and demonstrates irregular branching and arterio-venous shunts. Due to the unstable nature of these newly formed blood vessels, these vessels may be uniquely sensitive to ultrasound and may be significantly disrupted when exposed to low intensity ultrasound.

Ultrasound induced vascular disruption can occur by direct interaction between the ultrasound waves and the endothelial cell lining the vasculature. The heating and mechanical forces associated with ultrasound propagation may alter the cytoskeleton structure of the endothelial cells or dislodge the cells from their regular arrangement in the blood vessel lining to render the blood vessels leaky and ineffective for blood flow.

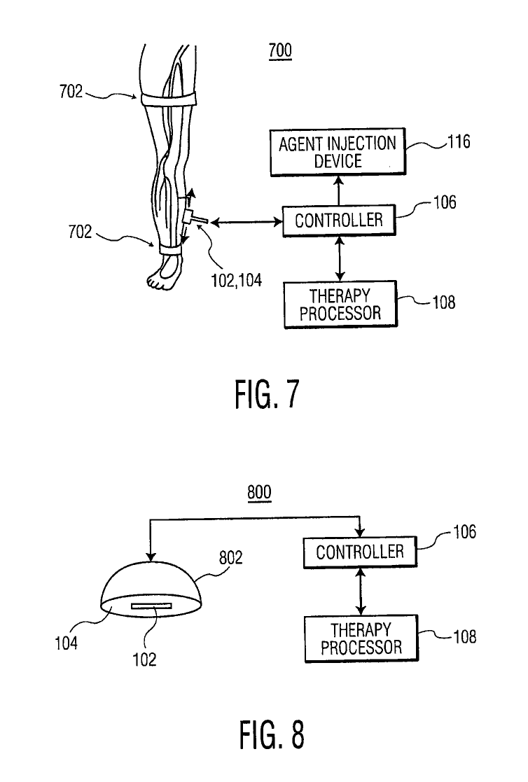

In addition, because system 100 targets blood vessels, system 100 may also be used to treat various vascular conditions, such as varicose veins (described further with respect to FIG. 7), macular degeneration (described further with respect to FIG. 8), chelolds, warts, fibroids, hemorrhoids, psoriasis and any other diseases and conditions mediated by angiogenesis. It is contemplated that system 100 may be used, for example, for cutaneous lesions, secondary tumors, metastases, physiotherapy, and tumor ablation in the brain, lungs and liver.

Therapy device 104 may include devices suitable for cutaneous and subcutaneous treatment. Non-limiting examples of therapy device 104 include wands, paddles, catheters, vaginal probes and rectal probes. The catheters may include, for example, general infusion catheters, site specific infusion catheters and circulatory bypass catheters. It is understood that therapy device 104 may be physically attached to the body or be held in place manually.

For cutaneous, subcutaneous and shallow lesions (including primary and secondary superficial melanomas, and cancers of the head and neck, thyroid, breast, and testis) configurations may include, for example, a disk shape that generates continuous ultrasound with highest possible sonication frequency in the frequency range 3-10 MHz. Because the attenuation of ultrasound increases with frequency, a lesion located deep in the abdominal cavity (liver, kidney, and pancreas) may be treated at lower frequencies, for example, from about 1 to 3 MHz. The use of lower frequency may insure better penetration of ultrasound without heating the intervening tissue. Heating by the propagating ultrasound wave may be further reduced by using long tone bursts of ultrasound waves instead of continuous waves. An alternate approach for treating deep lesions could also be to mount the therapy and imaging transducers 202, 204 on a laparoscope.

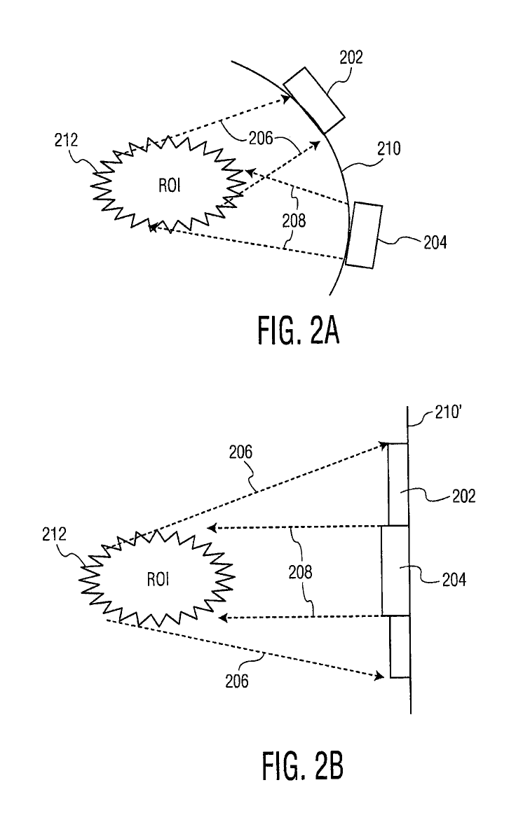

Therapy device 104 may include one therapy transducer elements or multiple transducer elements arranged in a linear, circular or nonlinear array. (A general therapy transducer 204 is shown in FIGS. 2A and 2B). Therapy device 104 may also include a beamformer and/or an amplifier (not shown), coupled to the transducer elements to insonate a target volume of the subject, within the ROI by a static or scanned ultrasound beam. Furthermore, therapy device 104 may include a processor (not shown) configured to insonate the target volume according to exposure conditions (described below), responsive to controller 106. It is understood that transducer elements of therapy device 104 may be enclosed in a jacket containing cooling fluid to avoid excessive heating by the transducer elements at a skin-transducer interface.

The exposure conditions represent some of the acoustic dosing conditions (also referred to herein as dosing conditions) monitored and adjusted by system 100. The exposure conditions may include, for example, an intensity of the LIU, a beam size of the LIU, a frequency of the LIU, a degree of focusing and whether the LIU is continuous or pulsed. The dosing conditions also include the treatment period, a duty cycle for the cessation periods between successive treatments in the therapy period, and a rate of infusion (described further below) of an agent used for imaging and/or for therapy.

The beam size may be used to control the area of the region to be sonicated. The LIU frequency may be selected, for example, based on the depth of the desired volume, with lower sonication frequencies typically being used for deeper penetration. The LIU intensity may be selected based on the area of the target, with larger areas and increased vascularity typically using a higher intensity (as well as a longer treatment period). The duty cycle may be selected to minimize tissue damage. In an exemplary embodiment, the LIU frequency is between about 20 kHz to about 20 MHz; the duty cycle is between about 0.1 to 1; and the treatment period is between a few seconds to about an hour. It is understood that any suitable dosing conditions may be selected which modify a target blood vessel without disrupting surrounding tissue.

Referring next to FIGS. 2A and 2B, functional block diagrams of configurations of imaging transducer 202 and therapy transducer 204 relative to ROI 212 are shown. FIG. 2A illustrates imaging transducer 202 and therapy transducer 204 located at different positions on assembly 210; and FIG. 2B illustrates imaging transducer 202 and therapy transducer 204 being collocated on assembly 210′. In FIGS. 2A and 2B, imaging transducer 202 represents one or more transducer elements of imaging device 102 and therapy transducer 204 represents one or more transducer elements of therapy device 104.

In FIG. 2A, imaging transducer 202 and therapy transducer 204 are each mounted on common assembly 210 and angulated such that imaging transducer 202 sonicates ROI 212 with energy 206, and therapy transducer 204 sonicates ROI 212 with energy 208. Accordingly, imaging transducer 202 and therapy transducer 204 sonicate the same ROI 212 from different viewing angles.

In FIG. 2B, imaging transducer 202 and therapy transducer 204 are collocated on common assembly 210′. Accordingly, in FIG. 2B, imaging transducer 202 and therapy transducer 204 sonicate the same ROI 212 from a same viewing angle. It may be appreciated that, in the exemplary configuration shown in FIG. 2B, imaging transducer 202 and therapy transducer 204 may be considered to represent one unit for imaging and therapy.

Referring back to FIG. 1, controller 106 is configured to control imaging device 102 and therapy device 104, as well as to control/implement therapy processor 108, and, optionally, infusion device 116. Controller 106 is also configured to receive user inputs from user interface 112, such as a ROI indicator and values for directing the applied treatment during the therapy period. Controller 106 is further configured to control the display of acquired images, including the ROI, an estimated vascularity, estimated changes of vascularity and/or dosing conditions on display 110. Furthermore, controller 106 may also control storing of acquired images, an estimated vascularity, estimated changes of vascularity and/or dosing conditions during the therapy period. Controller 106 may be a conventional digital signal processor. It will be understood by one of skill in the art from the description herein that one or more of the functions of therapy processor 108 may be implemented in software and may be performed by controller 106.

Therapy processor 108 is configured to receive images from imaging device 102 via controller 106, to estimate the change in vascularity of the target blood vessel and to determine whether to adjust at least one of the dosing conditions. Therapy processor 108 includes vascularity estimator 118 and acoustic dosing condition adjuster 120.

Vascularity estimator 118 receives the acquired images from imaging device 102 and estimates the change in vascularity from the received image. According to an exemplary embodiment, pixels associated with the target blood vessel may be identified from an initial image, prior to a first treatment period. An initial vascularity may be estimated from a ratio of a number of pixels (n) associated with the blood vessel and a number of pixels (N) in the ROI. The estimated vascularity (A) (as a percentage), for any treatment period, is given by eq. (2) as:

$$A = \frac{n}{N} \cdot 100 \qquad ( 2 )$$

The estimated vascularity represents an area of the ROI perfused with blood.

A further estimated vascularity may be determined after each treatment period. Accordingly, a change in vascularity (ΔA) (as a percentage) may be determined using the difference between estimated vascularities between adjacent treatment periods. The change in vascularity (ΔA) is shown in eq. (3) as:

$$Δ A = \frac{A ( post \text{-} treatment ) - A ( pre \text{-} treatment )}{A ( pre \text{-} treatment )} \qquad ( 3 )$$

The change in vascularity may be measured by ultrasound imaging or other suitable forms of imaging. To achieve a maximum sensitivity for contrast-enhanced ultrasound imaging, the imaging may be performed at low ultrasound exposure by either using a low frame rate or by using low mechanical index. A loss in vascularity can also be assessed by measuring the regional flow of an agent to the tissue, as described in U.S. Pat. No. 6,858,011 to Sehgal. Other dynamic imaging techniques, such as MRI, CT and PET, that measure blood flow and tissue vascularity may also be used to assess the therapeutic response and guide treatment.

If system 100 includes agent injection device 116, agent injection device 116 may be configured to direct an agent to the ROI. The injected agent may be directed at enhancing images and/or for therapy. It is understood that the agents used for imaging and therapy may include a same agent or different agents. Pixels enhanced by an agent used for imaging may be used to identify the pixels associated with the blood vessel. Agent injection device 116 may include an infusion pump, as well as a microprocessor (not shown). The infusion rate of the agent may be controlled by the microprocessor on the infusion pump, where the infusion pump receives instructions of the flow settings from controller 106.

Controller 106 receives the initial vascularity from vascularity estimator 118 to select initial dosing conditions. It is understood that initial dosing conditions may also be selected by acoustic dosing condition adjuster 120. According to an exemplary embodiment, a lookup table correlating percent response (reduced vascularity) and the treatment parameters (ultrasound intensity, treatment time, duty cycle, microbubble infusion rate) may be used for initial dosing conditions. The lookup table may be constructed either from clinical and/or preclinical studies or by numerical modeling of the tissues.

Acoustic dosing condition adjuster 120 receives the estimated change in vascularity from vascularity estimator 118 and determines whether to adjust at least one dosing condition. For example, the change in vascularity may be compared to a predetermined treatment response. If the change in vascularity is less than the predetermined treatment response, one or more dosing conditions may be adjusted. For example, the sonication intensity, treatment time and/or the rate of microbubble infusion may be increased. Any adjustments to the dosing conditions are provided by acoustic dosing condition adjuster 120 to controller 106.

User interface 112 may be used to initiate selection of a ROI, in order to determine an initial vascularity (to provide a ROI indicator). In addition, user interface 112 may be used to select values provided to therapy processor 108 for estimating vascularity and adjusting dosing conditions. User interface 112 may further be used to direct treatment during the therapy period, as well as to direct any images received from imaging device 102 to be displayed and/or stored. User interface 112 may include any suitable interface for initiating measurements, directing treatment and indicating storage and/or display of images. User interface 112 may also include an input device such as a keypad for entering information.

Display 110 may be configured to display one or more images including a respective ROI, as well as any dosing conditions, estimated vascularities and/or changes in vascularity during the applied therapy. It is contemplated that display 110 may include any display capable of presenting information including textual and/or graphical information.

Memory 114 may store images received from imaging devices 102, as well as estimated vascularities, estimated vascularity changes and/or dosing conditions from therapy processor 108. Memory 114 may also store information relating to the performed therapy such as the number of treatment periods and the duration of the therapy period, for example, for further analysis. It is understood that information stored in memory 114 may be used to modify a predetermined treatment response and/or a predetermined therapy response. Memory 114 may be a memory, a magnetic disk, a database or essentially any local or remote device capable of storing data.

It will be understood by one of skill in the art from the description herein that system 100 may be configured as a stand-alone portable device. It will also be understood by one of skill in the art from the description herein that imaging device 102 and therapy device 104 and, optionally, agent injection device 116 may be located remote from controller 106 and therapy processor 108, such as for remote measurements. Imaging device 102 and therapy device 104 may be connected to respective first and second terminals 122, 124 of controller 106 by any suitable connection. It will also be understood that controller 106 and/or therapy processor 108 may be located remote from display 110.

It is contemplated that system 100 may be configured to connect to a global information network, e.g., the Internet, (not shown) such that the received images, estimated vascularities, estimated changes in vascularity and/or the dosing conditions during the therapy period may also be transmitted to a remote location for further processing and/or storage. The connection may be by wire or may be a wireless connection.

System 100 may also include agent injection device 116. Agent injection device 116 is configured to direct an agent including through the bloodstream into blood vessels in the ROI. The agent may include suspensions of solid particles, emulsified liquid droplets and gas-filled bubbles, known as “microbubbles.” The agent (for example, Definity®, Lantheus, Medical Imaging, MA, USA) may be used with imaging device 102 to improve the quality of the acquired image. For example, the agent may intensify reflections of imaging ultrasound energy waves.

In an exemplary embodiment, an agent containing microbubbles may also be used with therapy device 104 to amplify the induced antivascular effect (i.e., vessel modification and/or disruption) during sonication. Referring to FIGS. 3A, 3B and 3C, diagrams of vascular system 300 are shown illustrating the effect of microbubbles 304 when insonified by therapy energy wave 208. In particular, FIG. 3A shows vascular system 300 infused with microbubbles 304 and insonated with therapy ultrasound wave 208; FIG. 3B shows that microbubbles 304′ interact with ultrasound wave 208 and undergo mechanisms such as forced oscillations and resonance; and FIG. 3C shows that endothelial cells 302 are modified and that vascular system 300 is disrupted.

Microbubbles 304, when injected intravenously, circulate in the intravascular space and are typically in the close proximity of a lining of endothelial cells 302. When vascular system 300 is sonicated with ultrasound wave 208, microbubbles 304 undergo forced oscillation, represented as microbubbles 304′ (FIG. 3B). Damping of these oscillations may dissipate acoustic energy to heat. Although the damping may occur through thermal, viscous and acoustic dissipation mechanisms, viscous damping due to high shell viscosity of microbubbles 304′ and the surrounding blood is typically a major source of damping and heat deposition.

There is a difference between direct heating by ultrasound and an indirect heating mediated by microbubbles 304. Direct heating occurs over the entire distance of ultrasound propagation, whereas bubble-mediated heating is localized and occurs at the sites where microbubbles 304 are present. Accordingly, microbubbles 304 not only act as transducers for converting acoustic energy to heat but they also tend to localize the delivery of acoustic energy to the targeted region (such as vascular system 300 and endothelial cells 302). In addition to heat conversion, microbubbles 304′ may also generate shear forces around their surface due to oscillation. Mechanical forces and heating by the microbubbles in the vicinity of endothelial cells 302 may damage the endothelial cells and disrupt the vascular structure of vascular system 300 (as shown in FIG. 3C).

The presence of microbubbles in a medium may induce inertial cavitation at lower sonication intensities. For example, in liquids, inertial cavitation has been observed at the pressure amplitude of 0.58 MPa in the presence of an agent and in rabbit ear vessels inertial cavitation activity has been reported at the pressure amplitude of 1.1 MPa using pulsed ultrasound in the presence of microbubble agents. If inertial cavitation does occur, it may also disrupt microvasculature and may also contribute to the antivascular activity, such as providing a reduced incidence of vascular disruption as the treatment frequency is increased. Because the interaction between ultrasound and microbubbles is complex and multifaceted, it is possible that with appropriate bubble distributions and sonication conditions, inertial cavitation and other nonlinear interactions may contribute to the antivascular activity.

In an exemplary embodiment, the microbubbles are less than about 8-10 μm in diameter, are stable structures and are able to pass through pulmonary circulation. In general, the microbubbles may be moieties/structures that encapsulate gas (which may be insoluble gas) within solid microshells. The encapsulated gas provides the microbubble with a high compressibility. The microshells may stabilize the microbubbles by preventing the gas from dissolving into the surrounding liquid. It is understood that the shells containing the gas are typically elastic (i.e., flexible) to undergo forced oscillations but also have a shear viscosity to cause viscous damping.

A size of the microbubble and the viscoelastic property of the encapsulating shell may be used to determine the resonance frequency of the microbubbles when driven by external ultrasound, such as by therapy device 104 (FIG. 1). For maximum transfer of acoustic energy to heat and shear waves, it is understood that the size and shell properties of the microbubbles are selected such that they are driven at resonance frequency. It is understood, however, that microbubbles will transfer acoustic energy to heat and shear even when not driven at resonance.

The microbubbles are desirably “endothelium-philic”. An affinity of the microbubbles for vascular endothelium may increase the contact time between the two entities and thus enhance the antivascular effect. This may be achieved by choosing a suitable shell material (for example, lipids, proteins, or polymers) and/or by attaching ligands on the shell surface that bind to molecular targets on the endothelium.

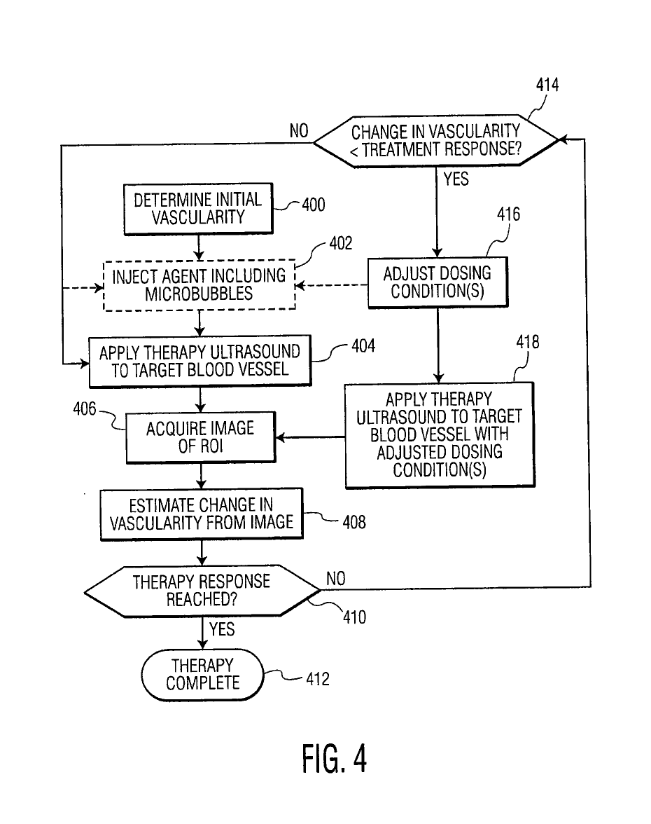

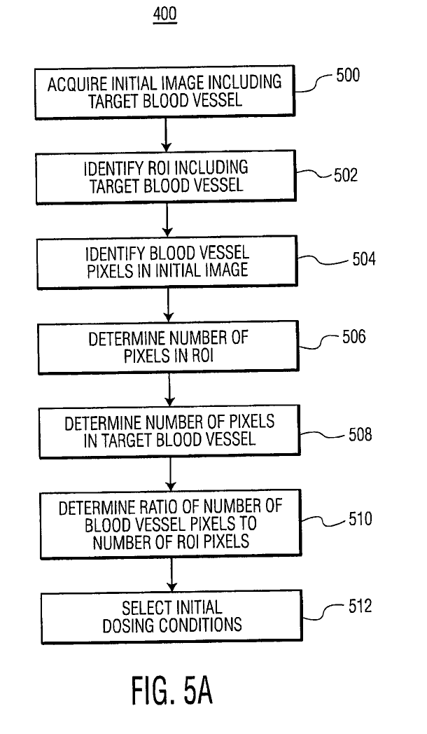

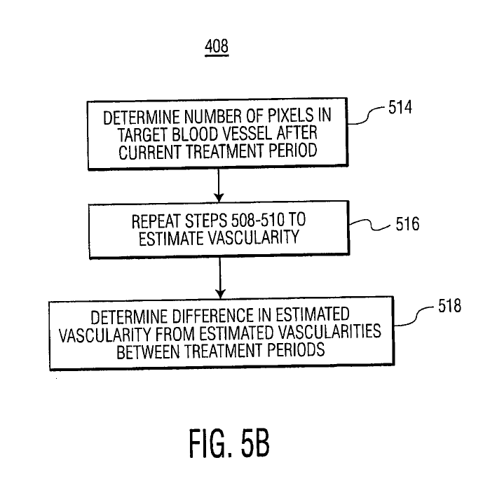

Referring next to FIG. 4, a flow chart illustrating an exemplary method for treating blood vessels is shown. At step 400, an initial vascularity is determined (described further with respect to FIG. 5A), prior to administering therapy. For example, the initial vascularity may be determined using imaging device 102 (FIG. 1), controller 106 and therapy processor 108. At optional step 402, a microbubble agent may be injected, for example by agent injection device 116 (FIG. 1) such that the microbubbles are directed to the target blood vessel within the ROI. Although not shown, step 402 may also include injecting an agent for imaging, for example by agent injection device 116 (FIG. 1).

At step 404, therapy ultrasound is applied to a target blood vessel in the ROI, for example, by therapy device 104 (FIG. 1) responsive to controller 106. At step 406, an image is acquired which includes the ROI, for example, by imaging device 102 (FIG. 1), responsive to controller 106. At step 408, a change in vascularity is estimated from the image acquired at step 406, for example, by vascularity estimator 118 (FIG. 1), further described with respect to FIG. 5B.

At step 410, it is determined whether a predetermined therapy response has been reached, for example, by controller 106 (FIG. 1), based on the estimated change in vascularity determined at step 408. If a predetermined therapy response is reached, step 410 proceeds to step 412 and the therapy is complete.

If the predetermined therapy response has not been reached, step 410 proceeds to step 414. At step 414, it is determined whether the estimated change in vascularity is less than a predetermined treatment response, for example, by acoustic dosing condition adjuster 120 (FIG. 1).

If a predetermined treatment response is greater than or equal to the predetermined treatment response, step 414 proceeds to step 404 (or to optional step 402), and steps 404 (or 402) through 410 are repeated.

If a predetermined treatment response is less than the predetermined treatment response, step 414 proceeds to step 416. At step 416, at least one dosing condition is adjusted, for example, by acoustic dosing condition adjuster 120 (FIG. 1). Step 416 may proceed to optional step 402, if one of the dosing conditions to be adjusted includes an infusion rate for the agent. At step 418, the therapy ultrasound is applied to the target blood vessel with the adjusted dosing condition. Step 418 proceeds to step 406, and steps 404 through 410 are repeated.

Because vessels may re-grow (for example through angiogenesis) after an applied therapy, it is contemplated that multiple therapies may be performed. The resumption in the growth of cancer vessels is likely to differ in individual patients and with the aggressiveness and the type of cancer. Therefore, the number of therapy sessions a patient receives may be determined on a case by case basis. The patients may be monitored by diagnostic contrast enhanced imaging on a regular basis. If the vessels begin to grow, the patient may receive another image-guided therapy, as described herein.

Referring to FIG. 5A, a flow chart illustrating an exemplary method of determining an initial vascularity (step 400 in FIG. 4) is shown. At step 500, an initial image is acquired including the target blood vessel, for example, by imaging device 102 (FIG. 1) responsive to controller 106. At step 502, an ROI is identified in the image, for example, using display 110 (FIG. 1) and user interface 112. At step 504, the target blood vessel is identified within the ROI, for example, using display 110 (FIG. 1) and user interface 112.

At step 506, a number of pixels (N) in the ROI is determined, for example, by vascularity estimator 118 (FIG. 1). At step 508, a number of pixels (n) of the target blood vessel is determined, for example, by vascularity estimator 118 (FIG. 1).

At step 510, a ratio of the number of blood vessel pixels (n) to the number of ROI pixels (N) is determined, for example, by vascularity estimator 118 (FIG. 1). At step 512, initial dosing conditions are selected, for example, by acoustic dosing condition adjuster 120 (FIG. 1) or controller 106.

Referring to FIG. 5B, a flow chart illustrating an exemplary method of estimating a change in vascularity (step 408 in FIG. 4) is shown. At step 514, a number of pixels in the target blood vessel is determined after a current treatment period, for example, by vascularity estimator 118 (FIG. 1). At step 516, steps 508-510 (FIG. 5A) are repeated to determine an estimated vascularity after the current treatment period. At step 518, a difference in the estimated vascularity is determined from the estimated vascularities before and after the current treatment period (i.e. between adjacent treatment periods), for example, by vascularity estimator 118 (FIG. 1). The difference in vascularity represents the change in vascularity for the current treatment period.

Referring back to FIG. 1, the present invention may provide advantages, such as the ability to produce clinical effects within a tumor without damaging the surrounding tissue. Exemplary system 100 uses LIU, with an intensity comparable to that used in physiotherapy, but considerably lower than the intensity of HIFU treatment of cancers. In general, system 100 may be less costly, simpler to design and easier to use in clinical settings as compared to conventional HIFU systems. System 100 may perform therapy ultrasound without using large acoustic windows in the body to achieve large focal gains of acoustic pressure. Entire regions of tissues can be treated directly with the unfocussed or mildly focus beams of therapy device 104 without the need to ‘paint’ the lesion by successive small regions of treatment.

To focus ultrasonic energy to specific region in conventional HIFU treatments requires knowledge of tissue parameters, which typically cannot be measured or predicted with high accuracy. System 100 overcomes this problem by monitoring the treatment in real time; by imaging and controlling the treatment with a feedback loop (i.e., by controller 106) to control treatments. System 100 does not use a priori information regarding tissue properties. In addition, the antivascular activity produced by system 100 occurs at the sites where ultrasound propagation intersects with the passage of microbubbles moving slowly through the blood vessels.

The high intensity fields used in conventional HIFU may coagulatively necrose or cauterize tissues. The acoustic impedance mismatch associated with the tissue changes prevents the subsequent transmission of ultrasound along the depth and makes the treatment of the region beyond the focal region difficult to achieve. As a result of this limitation, in conventional HIFU, the distal lesion is treated first before treating the proximal lesion. In the event that a part of the lesion is incompletely treated, it can not be subsequently accessed. System 100 of the present invention does not cause coagulative necrosis or cauterization of the tissue and, thus, the lesion can be treated repeatedly.

Another advantage of the present invention is that system 100 does not require treatment of individual cancer cells. Because the survival of several thousand cells depends on every individual blood vessel, disrupting a few blood vessels may trigger cell death in many cancer cells.

A further advantage of the present invention is that the target body structure, the endothelial cells, are in close proximity of the microbubbles. Because of the easy access to the target body structure, system 100 is not limited by drug delivery problems common with therapies which target cancer cells in the extravascular space. Furthermore, system 100 uses access to the surface of the endothelial cells, unlike other antivascular drugs that need to penetrate the cells to affect their cytoskeleton.

Another advantage of the present invention is that, unlike antivascular compounds that target molecular pathways or molecular events specific to individual tumor types, system 100 targets endothelial cells present in all tumor types and, therefore, may have a general applicability to any type of tumors. Furthermore, the present invention makes it feasible to treat tumors locally and may not cause side effects and/or drug resistance often associated with systemic treatments with chemotherapeutic and other molecular agents.

According to another embodiment of the present invention, an agent using microbubbles may be used with LIU to limit blood flow to and from the tumor, and retain a therapeutic agent within the tumor. For therapeutics to be effective, the agents are transported from the capillaries to the interstitial space. The trans-capillary flow is determined by the hydrostatic and colloid osmotic pressure difference between the intravascular pressure and the interstitial fluid pressure (IFP). In normal tissue there is net outward filtration pressure of about 1-3 mm. In tumors there is an increase in microvessel density and the individual blood vessels are not well formed and leak excess fluid to the interstitial spaces. Due to a poor or non-existent lymphatic system, within the cancer mass excess fluid is not drained and as a result fluid accumulates in the stroma, leading to local hypertension. A build up of high IFP that equals or exceeds the intravascular pressure inhibits the outflow of cancer drugs from capillaries to the extravascular space surrounding the cancer cells.

To increase the drug uptake several pharmaceutical agents are being developed to reduce the fluid pressure in the interstitium. According to an embodiment of the present invention, LIU in combination with microbubbles may disrupt tumor microvessels. This ultrasound vascular disruption may be used as a vehicle for improving drug delivery by trapping the drugs in a cancer volume.

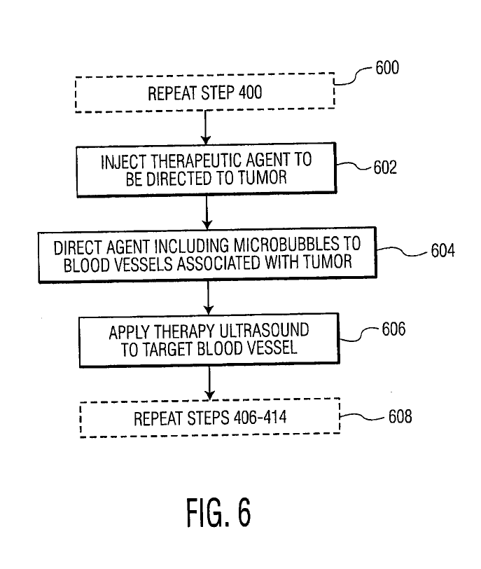

Referring to FIG. 6, a flow chart illustrating an exemplary method of treating a tumor with a therapeutic agent (referred to herein as sonic trapping) is shown. At optional step 600, step 400 (FIG. 4) may be repeated to determine an initial dosing condition. At step 602, a therapeutic agent is introduced into the bloodstream to be directed to a tumor. The delivery of the therapeutic agent may be intravascular or oral.

At step 604, an agent including microbubbles is directed to the tumor, for example, by agent injection device 116 (FIG. 1). At step 606, therapy ultrasound is applied to the target blood vessel such that the microbubbles interact with the therapy ultrasound to disrupt at least one of the blood vessels, for example, using therapy device 104 (FIG. 1) with LIU. The drug content of the tumor vessels are dispersed in the tumor interstitium.

It is preferable that the ultrasound antivascular treatment is applied (step 604) when the therapeutic agent achieves its maximum concentration in the bloodstream. At optional step 608, steps 406-414 (FIG. 4) may be repeated, if additional treatment periods are used in the therapy period.

With no blood flow and lack of lymphatic drainage, the cancer drug is trapped within the cancer mass until the new blood vessels develop through angiogenic growth. The delivery method is independent of interstitial fluid pressure. The exemplary sonic trapping method would not have side effects commonly associated with systemic use of drugs for reducing IFP. By sonic trapping, it may be feasible to reduce concentration of cancer drugs which usually have high toxicity. Sonic trapping may also be used to provide a locally high concentration of the drug in the tumor. Although sonic trapping is illustrated in FIG. 6 with respect to a tumor, sonic trapping may be useful for treating any disease and/or condition mediated by angiogenesis.

The exemplary sonic trapping method may be useful in enhancing the efficacy of chemotherapeutic agents. Low doses of the chemotherapeutic agent could be delivered to the cancer site through intravenous injection or oral ingestion. The circulating agent could be trapped in the cancer by the exemplary antivascular ultrasound treatment method. Because cancers do not generally have developed lymphatic system, the chemotherapeutic agent may be trapped in the tumor until there is new growth of blood vessels. Patients who undergoing this combined chemotherapy—sonic trapping treatment may be monitored routinely for the new growth of blood vessels by ultrasound contrast imaging or other forms of imaging. In the event that a new growth of blood vessels is observed, the chemotherapeutic/sonic trapping method described above may be repeated.

In addition to cancer treatments as described above, exemplary antivascular treatment with low intensity ultrasound may also be used, for example, for treating varicose veins, macular degeneration, cheloids/warts fibroids, hemorrhoids, psoriasis or other conditions affects by angiogenesis. Furthermore, the exemplary sonic trapping method may be suitable for enhancing the efficacy of chemotherapeutic agents. Although the general approach for various treatments is similar, measures specific to applications described below may also be taken during treatment.

Referring to FIG. 7, a perspective view of an exemplary system 700 for treating varicose veins is shown. System 700 includes imaging device 102 and therapy device 104, shown here as one device. System 700 also includes, pressure cuffs 702 (or tourniquets), controller 106, therapy processor 108 and agent injection device 116. Although not shown, system 700 may also include components such a user interface, a display, and a memory, as described above with respect to FIG. 1.

The antivascular ultrasound treatment can be used to treat varicose veins, the enlarged twisted veins that commonly appear raised above the surface of the skin on the inside of the leg or on the backs of the calves. The treatment would consist of injecting microbubble agents, using agent injection device 116 followed by ultrasound treatment (as described herein), and using imaging device 102 and therapy device 104. Although ultrasound frequencies of 0.5 to 3 MHz may be used for treatment, higher frequencies (>5 MHz) for treatment may also be suitable to prevent penetration of ultrasound deep into the tissue. Before insonation, but after contrast injection, blood flow through the vein may be reduced by pressure cuffs 702, to increase the time of interaction between the ultrasound and microbubbles.

Common interventional treatments consist of surgical stripping of the sephenous vein or nonsurgical therapy by endovenous laser or radiofrequency treatments. Unlike these methods, system 700 will not involve any interventional procedures.

Referring to FIG. 8, a perspective view of an exemplary system 800 for treating macular degeneration is shown. System 800 includes imaging device 102, therapy device 104 and enclosure 802. System 800 also includes controller 106 and therapy processor 108. Although not shown, system 800 may also include components such a user interface, a display, and a memory, as described above with respect to FIG. 1.

The exudative (wet) form of macular degeneration is often caused by abnormal blood vessel growth from the choroid behind the retina. Injection of anti-angiogenic drugs in the vitreous humor of the eye has been proposed to improve the vision. The injections are costly, painful and must be repeated frequently (bi-weekly). System 800 which uses antivascular ultrasound as described above has potential for treating macular degeneration. Due the close proximity to the surface and low attenuation of ultrasound by the eye tissue, intensities lower than those used for treating cancer may be useful. The therapy device 104 may be enclosed in a cup-shaped enclosure 802. An imaging transducer of imaging device 102 and/or therapy transducer of therapy device 104 may include a concave shape that conforms to the geometry of the eye.

The present invention is illustrated by reference to several examples. The examples are included to more clearly demonstrate the overall nature of the invention. The examples are exemplary, and not restrictive of the invention.

### Example 1

### Effect of Antivascular Therapy on Survival Time

To determine whether the antivascular effects of ultrasound improve the survival rate, thirteen animals with melanoma implanted subcutaneously were studied. The animals were randomly divided into two groups: a control group and a test group. In the test group, 8 animals received one 3 minute treatment with 3 MHz ultrasound at 2.3 W/cm². In the control group, the remaining 5 animals did not receive any treatment. The growth of tumors in all the animals was determined by measuring the tumor size with ultrasound imaging. The size was measured approximately every two days. The time to reach tumor size of 3 ml was used as the endpoint for the survival time.

The volume (mean±standard deviation) of the tumor on the treatment day for the control and test groups was 873±386 mm³, and 700±211 mm³. A two tailed Student's t-test showed the difference in volume for the two groups to not be significant (p≦0.394).

Acute change in tumor volume as a result treatment was observed in the test group (n=7) on the day of the treatment. Due to scattered intercellular edema, the volume of the tumor increased from the pre-treatment value of 669±249 mm³ to the post treatment value of 894±295 mm³. The difference between pre-treatment and post treatment values was 225±199 mm³; the difference is significant (2-tailed paired t test, p≦0.024).

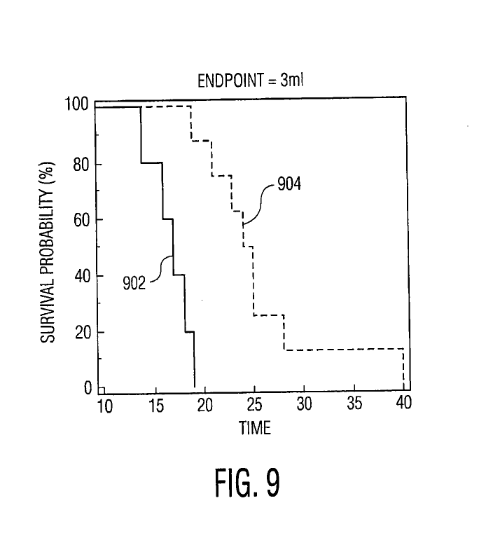

Referring to FIG. 9, a graph illustrating an example of survival probability as a function of time with application of an antivascular therapy, according to an embodiment of the present invention, is shown. FIG. 9 shows the Kaplan-Meier curves for the control group 902 and the treated group 904. The median survival time increased from 17 days for the control group to 24.5 days for the test group that received the treatment. The difference in the survival time is significant (p=0003, hazard ratio 5.126, 95% confidence level 5.224 to 268.48). In summary, a single treatment of the tumor with antivascular ultrasound increased the survival significantly.

### Example 2

### Effectiveness of Antivascular Ultrasound Therapy

Longitudinal studies in mice with implanted tumors were performed to evaluate the effectiveness of antivascular ultrasound therapy. The animal studies were performed in 32 mice (6 to 8 weeks of age; C3HV/HeN strain), randomly placed into treated (n=15) or control (n=17) groups. In each mouse two million murine melanoma cells (K1735²²) were injected subcutaneously in the right flank. About a week later the mouse was anesthetized with isoflurane and oxygen, and the hair coat overlying the injection site was removed by clipping and applying a depilation cream. As soon as the tumor was visually detected, the mouse was re-anesthetized and a B-mode ultrasound examination was performed (7-15 MHz broad-band probe). In each of two orthogonal B-mode images, the length (L), width (W) and depth (D) of the tumor was measured and its volume (ml) was calculated by the formula V=0.5 LWD, where D was measured in the two image planes and averaged. Each mouse was then re-anesthetized every two to three days and the tumor volume was again measured. Once the tumor had grown to about 1 ml in volume, a catheter was inserted into the tail vein, the mouse was anesthetized as described above, and 0.2 ml microbubble-containing, ultrasound contrast agent was injected. The contrast agent was injected in both control and treated groups. A sonographer making the tumor volume measurements and the contrast injection was blinded to the control and the treated group.

In the treated group, tumor therapy was performed with low intensity (e.g., about 2.4±0.1 W·cm⁻²) continuous 3 MHz ultrasound. Therapy commenced within two minutes of the completion of the injection of the contrast agent. Three one minute treatments were given with a one minute gap between each treatment (to ensure that the face of the probe remained cool, it was placed in ice water during the gap time). In the control group of mice, the physiotherapy ultrasound probe was applied to the tumor as described above, but the apparatus was not turned on (i.e., a sham treatment was performed).

B-mode ultrasound measurements of the growth of the tumor continued every two or three days. Once the tumor reached about 3 mL in volume, corresponding to about 10% body weight, each anesthetized mouse was euthanized by cervical dislocation. The time of euthanasia was used in plotting the survival curves.

In each mouse, the time (in days) from the injection of cancer cells to the first visual detection of a tumor was recorded, and expressed as a mean and standard deviation across all mice. In the control and treated groups, the tumor size on the day of treatment and day of euthanasia was recorded and expressed as a mean±standard deviation. A two-tailed T-test (for example, using MedCalc Software, Marlakerke, Belgium) was performed to look for differences in tumor volume between the two groups. A P-value of ≦0.05 was considered to be statistically significant. The time (in days) from the first measurement of the tumor size until euthanasia was recorded for each animal, and the percentage of animals surviving with time was plotted. A log rank test was used to analyze differences between the two survival curves, with 95% confidence limits also being calculated.

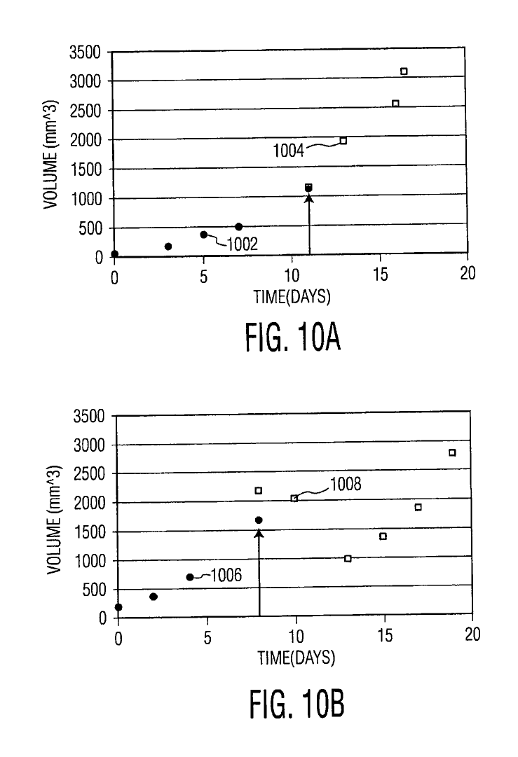

Referring to FIGS. 10A and 10B, graphs illustrating tumor growth as a function of time for the control group (FIG. 10A) and the treated group (FIG. 10B) are shown. In FIG. 10A, circles 1002 represent tumor volume of the control group before therapy and squares 1004 represent tumor volume following the sham treatment. In FIG. 10B, circles 1006 represent tumor volume of the treated group before therapy and squares 1008 represent tumor volume following the antivascular treatment. In FIGS. 10A and 10B, the arrow indicates the day of the therapy.

FIGS. 10A and 10B indicate that at about 10.3±4.7 days after the injection of cells, the tumor was visually detected and its volume was easily measured in the B-mode ultrasound examinations. The tumor was hypoechoic to the surrounding tissues and had distinct margins. The mice recovered normally from each of the general anesthetics. At the time of therapy, there was no statistically significant difference (P of about 0.36) between the sizes of the tumors of the treated and control groups of mice. For the treated group, the tumor volume was about 0.88±0.38 ml. For the control group, the tumor volume was about 1.00±0.35 ml.

As shown in FIG. 10B, in the treated mice, there was an increase in tumor volume immediately following therapy. Throughout the duration of the experiment, the mice exhibited normal behavior (i.e., each was active and ate and groomed itself normally). At euthanasia, there was no statistically significant difference (P of about 0.67) between the size of the tumor in the treated and control groups. For the treated group, the tumor volume was about 2.88±0.52 ml. For the control group, the tumor volume was about 2.78±0.54 ml.

In two mice, a significant cutaneous ulcer developed on the surface of the tumor and euthanasia was performed prior to the tumor reaching 3 ml in size (mouse A from the treated group, having a tumor volume at euthanasia of about 2.1 ml; and mouse B from the control group, having a tumor volume at euthanasia of about 1.2 ml). The growth of the tumors continued after treatment in four mice. In the remaining 11 mice, tumor growth decreased immediately after therapy but later resumed. There was no such interruption to growth in the tumors of the sham-treated mice (FIG. 10A). One mouse from the control group had a continued tumor growth following the sham-treatment. One mouse from the treated group had an immediate increase in tumor volume followed by a decline in tumor volume over the next five days, followed by a subsequent increase in tumor volume.

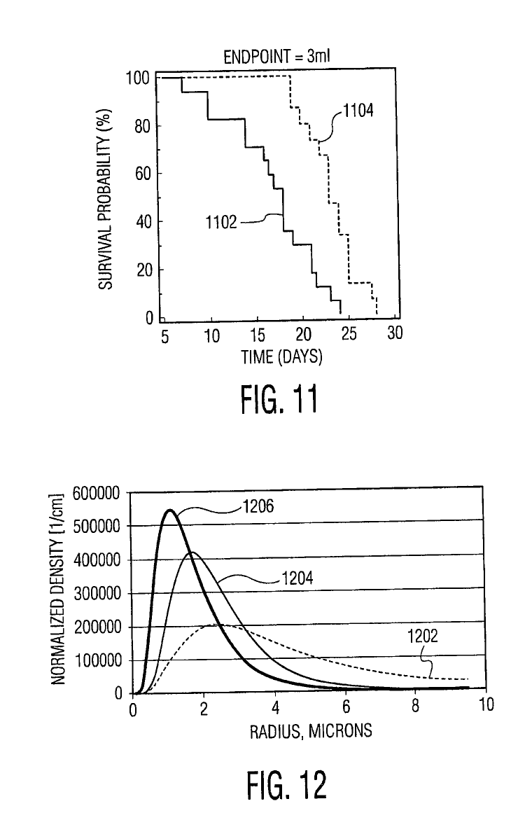

Referring to FIG. 11, a graph illustrating survival probability as a function of time is shown. In FIG. 11, curve 1102 represents the control group and curve 1104 represents the treated group. The median survival time for the treated group was about 23 days and for the control group was about 18 days. The 28% increase in survival time for the treated group was shown to be statistically significant (P≦0.0001). The 95% confidence interval was 2.5 to 14.7. At necropsy, all tumors had distinct boundaries and there was no evidence of local invasion.

In this example study of the growth of a primary cancer, it was demonstrated that animal survival time was increased by a single three minute episode of antivascular ultrasound treatment. Such a finding has not been reported following therapy with conventional combretastatins, another form of a tumor vascular disrupting agent. It is probable that the increased survival time found in this study was related to the disruption of the tumor vasculature, formed as a result of angiogenesis. Accordingly, ultrasound antivascular therapy may have future clinical potential for improving survival time for patients with cancer.

### Example 3

### Numerical Simulation of Ultrasound Heating in the Presence of Microbubbles

As discussed above, microbubbles may enhance the thermal effects of ultrasound therapy and may have a dominant role in disrupting the tumor neovasculature. In this example, computer simulations are performed, to assess the role of microbubbles in enhancing tissue heating. Because blood perfusion rate, heating rate (the product of ultrasound intensity and sonication time) and sonication frequency may be related to the thermal dose delivery, their potential roles are also studied. The approach, in this example, is to vary each of the parameters systematically and evaluate the heating response.

Heat deposition by oscillating microbubbles is a function of their equilibrium radius and the incident sonication frequency. In this example, the equilibrium radii of a contrast agent present in an animal's blood pool is modeled to be distributed over a range of values described by a probability density function. A lognormal distribution (N(R₀)) of microbubbles with equilibrium radii of R₀, shown in eq. (4) below, is assumed for the microbubbles.

$$N ( {R}_{0} ) = \frac{{N}_{T} exp ( - \frac{{ln}^{2} ( {R}_{0} / {R}_{pk} )}{4 {σ}^{2}}}{2 \sqrt{π} {R}_{p k} σ exp ( {σ}^{2} )} \qquad ( 4 )$$

In eq. (4), Rₚₖ represents the peak density radius, σ represents the standard deviation of the microbubble radii and N_{T} represents the total number of microbubbles per unit volume.

Ultrasonic absorption (α_{mb} db/cm) by an ensemble of microbubbles due to viscous damping of bubble oscillations (induced by ultrasonic vibrations at frequency ƒ) is related to the complex compressibility (β), the density (ρ) and the sound speed (c) of the microbubble suspension, as shown eq. (5) below.

$${α}_{mb} = 2 {log}_{10} e \cdot Im \{ \frac{2 π f}{c} \sqrt{1 + ρ {c}^{2} β} \} \qquad ( 5 )$$

In eq. (5), β is related to probability density distribution (eq. (4)), the total normalized damping constant (ζ_{T}) and the normalized resonance frequency ( ƒ̅) of the bubbles by eq. (6) as:

$$β = {\int }_{0}^{∞} \frac{{R}_{0} N ( {R}_{0} ) d {R}_{0}}{ρ {f}^{2} ( f + j {ζ}_{T} )} \qquad ( 6 )$$

Eqs. 4-6 are described in Razansky et al., “Enhanced heat deposition using ultrasound contrast agent-modelling and experimental observations,” IEEE Trans. Ultrasound Ferroelectric Frequency Control, January 2006, vol. 53, pp. 137-147, the contents of which are incorporated herein.

Because shell viscous damping (ζₛₕ) may be a major contributory damping mechanism for microbubble of the contrast agent, the total normalized damping constant (ζ_{T}) is assumed to be equal to the shell viscous damping (ζₛₕ).

The tissue temperature T during heating is calculated by using ultrasound absorption with eq. (7) (the bio-heat transfer equation):

$$ρ {C}_{t} \frac{\partial T}{\partial t} = κ {∇}^{2} T - {w}_{b} {C}_{b} ( T - {T}_{e} ) + Q \qquad ( 7 )$$

where Cₜ and C_{b} represent the respective specific heat of the tissue and blood (e.g., both equal to about 3770 J/kg/° C.), κ represents the thermal conductivity of tissue (e.g., about 33.6 J/min/m/° C.), Tₑ represents the equilibrium tissue temperature (e.g., about 37° C.), w_{b} represents the blood mass flow rate per unit tissue volume and Q represents the power deposited per unit tissue volume. For a plane wave of intensity I propagating along the z-axis in tissue with ultrasound absorption coefficient, α, the power Q may be represented by eq. (8) as:

Q=2αI(z)=2αI₀ exp(−2αz). (8)

Eq. (7) is solved for T for plane wave propagation. The total absorption coefficient of tissue with contrast agent, α in eq. (8), is taken as the sum of the absorption coefficient of tissue (e.g., about 0.04 Np/cm/MHz) and the absorption coefficient of contrast agent microbubbles (eq. (5)).

The thermal effects produced by three different commercial contrast agents are studied. The contrast agents included Optison™ (GE HealthCare, Chalfont St Giles, UK), Definity and Albunex® (Mallinckrodt Inc., Folcroft, Pa., USA) are studied. Referring to FIG. 12, a graph is shown illustrating the normalized density distribution of microbubbles in these three indicated contrast agents as a function of radius. In FIG. 12, curve 1202 represents the density with Definity, curve 1204 represents the density with Optison and curve 1206 represents the density with Albunex. The lognormal distributions of these microbubbles are constructed based on information published information from the manufacturers of the respective contrast agents. Although there are significant differences in the density distribution of the microbubbles of the various contrast agents, the different contrast agents, in general, produce similar thermal effects. Accordingly, only the thermal effects associated with Definity are discussed below.

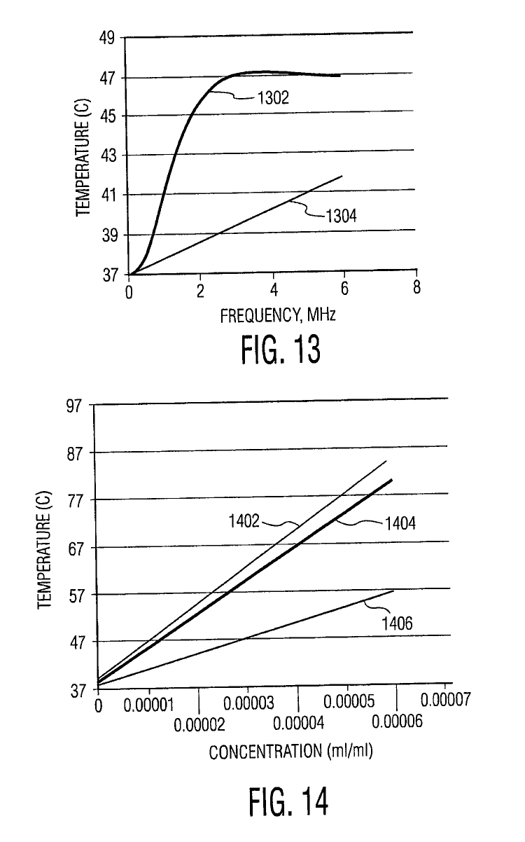

Referring to FIGS. 13 and 14, graphs are shown which illustrate the change in temperature due to presence of microbubbles. In particular, FIG. 13 shows the effect of temperature as a function of frequency due to ultrasonic heating in the presence of microbubbles; and FIG. 14 shows the effect of temperature as a function of concentration with application of sonication at various frequencies. In FIG. 13, curve 1302 represents the change in temperature due to ultrasound heating in the presence of a concentration of 10⁻⁵ (ml/ml) microbubbles with Definity; and curve 1304 represents the change in temperature due to ultrasound heating without microbubbles. In FIG. 14, curves 1402, 1404 and 1406 represent the change in temperature for microbubbles with Definity at ultrasound frequencies of 3 MHz, 2, MHz and 1 MHz, respectively. In FIGS. 13 and 14, the sonication time is 1 minute, with an ultrasound intensity of 2.2 W/cm².

As shown in FIG. 13, during sonication, significantly higher temperatures are achieved in the presence of a 10⁻⁵ (ml/ml) concentration of microbubbles than without microbubbles. As shown in FIG. 14, the enhancement in heating by microbubbles may increase with sonication frequency. Accordingly, antivascular action at 3 MHz may be greater than antivascular action at 1 MHz sonication. After reaching a maximum at 3 MHz, the acoustic-to-heat conversion may decrease with further increases in frequency. As shown in FIG. 14, the temperature increased linearly with microbubble concentration.

Although it may not presently be feasible to determine the local concentrations of contrast agent in the vasculature, the simulations shown in FIGS. 13 and 14 suggest that microbubbles, even when present in relatively small amounts, may be very effective in local heating of a specific region.

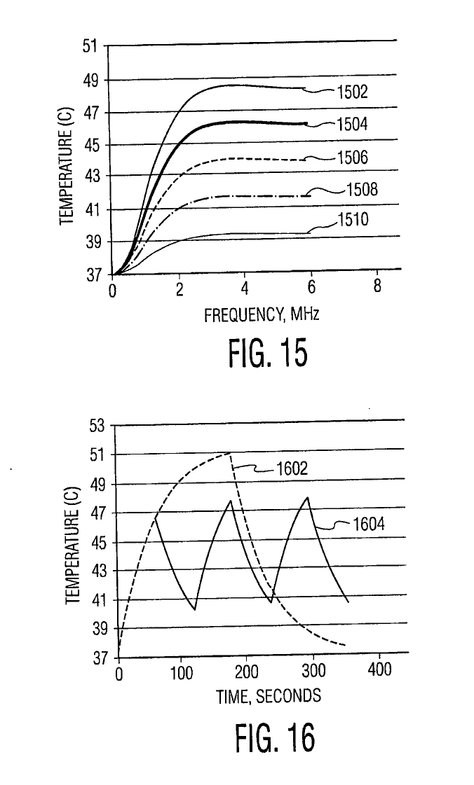

Referring to FIG. 15, a graph illustrating microbubble-induced heating as a function of frequency with various ultrasound intensities is shown. In FIG. 15, curves 1502, 1504, 1506, 1508 and 1510 represent heating at respective intensities of 2.5 W/cm², 2 W/cm², 1.5 W/cm², 1 W/cm² and 0.5 W/cm². FIG. 15 illustrates the effects of different intensities on the heating temperature. In FIG. 15, the sonication time is 1 minute for microbubbles at a concentration of 10⁻⁵ (ml/ml) with Definity. Higher temperatures are shown as the ultrasound intensity increased. The results indicate that by choosing appropriate intensity, it may possible to achieve desired temperatures with microbubble induced heating.

Referring to FIG. 16, a graph illustrating a change in temperature as a function of time with the application of continuous and intermittent sonication is shown. In FIG. 16, curve 1602 represents continuous sonication and curve 1604 represents intermittent sonication. The continuous sonication represents 3 minutes of sonication followed by 3 minutes of no sonication. The intermittent sonication represents three one-minute cycles of sonication followed by 1 minute of no sonication. In FIG. 16, a concentration of 10⁻⁵ (ml/ml) microbubbles with Definity is used. The sonication frequency is 3 MHz and the intensity is 2.2 W/cm². FIG. 16 illustrates the heating effects as a function of duty cycles with variable on and off intervals. Although the total exposure to ultrasound is the same for the two cases, intermittent heating may lead to lower temperatures.

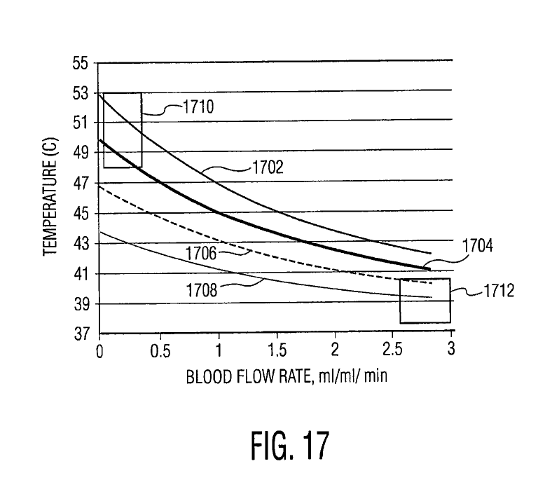

Referring to FIG. 17, a graph illustrating a change in temperature as a function of blood flow rate for various fractional interaction times is shown. Fractional interaction time refers to the ratio of time microbubbles are in the ultrasound field to the time ultrasound is on. In FIG. 17, a concentration of 10⁻⁵ (ml/ml) microbubbles with Definity is used. The sonication frequency is 3 MHz and the intensity is 2.3 W/cm². In FIG. 17, curves 1702, 1704, 1706 and 1708 represent the temperature change at respective fractional interaction times between the microbubbles and the ultrasound of 1, 0.75, 0.5 and 0.25.

Blood perfusion rates (w_{b}) may critically affect the thermal dose delivered to the tissue (eq. (7)). Higher perfusions may reduce thermal dose. On the other hand, higher perfusion may increase heating due to increased contrast agent delivery. It is possible that one or both of these factors may dominate the temperature change. Simulation studies are performed to calculate the temperature change at different perfusion rates (i.e., blood flow rates). In FIG. 17, a fixed perfusion rate is assumed during the sonication time.

FIG. 17 shows that higher perfusion rates may produce lower temperatures and may reduce the heating. The bioheat transfer equation (eq. (7)) only considers the effect of flow on dissipating heat from a volume element. It is also possible that flow may determine the time ultrasound and microbubbles interact to heat the local region. If the flow rate is slower, the microbubbles may be exposed to ultrasound longer than for a faster flow rate. In the limiting case, when there is no flow, the time that microbubbles are exposed to ultrasound may be equal to the time that the ultrasound is applied. That is, the fractional interaction time (i.e., the ratio of time microbubbles are in the ultrasound field to the time ultrasound is on) will be 1, or 100%. In contrast to the stationary limiting condition, when the flow rate is very fast, the individual microbubbles travel through the exposed volume very quickly without being significantly exposed to ultrasound. In this condition, the fractional interaction time approaches zero and the microbubbles do not significantly transform acoustic energy to heat. For intermediate flow rates, the interaction time may be between 0 and 1, and the heating of the tissue may be between the two limiting conditions (i.e., no flow to very high flow).

FIG. 17 shows that the extent of localized heating may be determined by the local flow rate. The implication of these results for cancer therapy is that, all other factors being equal, the heating pattern in tumor vessels (which are known to have sluggish flow) will correspond to the results shown in box 1710, while normal blood vessels with faster blood flow will follow the heating pattern in box 1712. That is, for the same ultrasound exposure, the temperatures achieved in abnormal blood vessels with sluggish blood flow may be much higher than those in the normal vessels. This temperature differential may make it possible to selectively disrupt tumor blood vessels without causing significant adverse effects on normal blood vessels.

The Example 3, described above, illustrates a methodology to simulate diverse conditions of microbubble-Induced heating. Data generated from these simulations may be useful in guiding vascular therapy and for planning individual patient treatment. According to aspects of the present invention, sonication time and sonication intensity may be adjusted, using a simulation model, to compensate for differences in the perfusion rates. For example, the model may provide information as to whether tumors with high perfusion rates should receive aggressive treatment (e.g., a higher sonication intensity, a longer treatment time, etc.).

Although the invention has been described in terms of systems and methods of treating blood vessels and treating a tumor with a therapeutic agent, it is contemplated that one or more steps and/or components may be implemented in software for use with microprocessors/general purpose computers (not shown). In this embodiment, one or more of the functions of the various components and/or steps described above may be implemented in software that controls a computer. The software may be embodied in non-transitory tangible computer readable media (such as, by way of non-limiting example, a magnetic disk, optical disk, hard drive, etc.) for execution by the computer.

Although the invention is illustrated and described herein with reference to specific embodiments, the invention is not intended to be limited to the details shown. Rather, various modifications may be made in the details within the scope and range of equivalents of the claims and without departing from the invention.

What is claimed:

1. A method of treating at least one blood vessel in a region of interest, the method comprising:
applying therapy ultrasound to the at least one blood vessel within the region of interest using one or more dosing conditions;
acquiring an image of the region of interest responsive to the applied therapy ultrasound; and
estimating a change in vascularity of the at least one blood vessel, responsive to the applied therapy ultrasound, using the acquired image to determine whether to adjust at least one of the dosing conditions,
wherein the therapy ultrasound is applied with an intensity to modify the at least one blood vessel without damaging a surrounding tissue.

2. The method of claim 1, the method further including, prior to applying the therapy ultrasound:
directing an agent including microbubbles to the at least one blood vessel in the region of interest,
wherein the microbubbles modify the at least one blood vessel responsive to the therapy ultrasound.

3. The method of claim 1, wherein the one or more dosing conditions includes at least one of an exposure condition of the therapy ultrasound or a treatment period for applying the therapy ultrasound.

4. The method of claim 1, wherein the intensity of the therapy ultrasound is less than or equal to about 5 W/cm².

5. The method of claim 1, further including:
repeating the applying of the therapy ultrasound, the acquiring of the image and the estimating of the change in vascularity until the estimated change in vascularity corresponds to a predetermined treatment response.

6. The method of claim 1, further comprising, prior to the applying of the therapy ultrasound:
acquiring an initial image including the region of interest;
selecting the region of interest from the initial image; and
determining the one or more dosing conditions from the selected region of interest within the initial image.

7. The method of claim 6, wherein the estimating of the change in vascularity includes:
estimating an area of the selected region of interest perfused with blood from the initial image to form a first vascularity;
estimating an area of the region of interest perfused with blood from the acquired image to form a second vascularity; and
determining a difference between the second vascularity and the first vascularity to form the estimated change in vascularity.

8. The method of claim 1, wherein the at least one blood vessel is associated with a tumor.

9. A method of disrupting at least one blood vessel in a region of interest, the method comprising:
a) directing an agent including microbubbles to the at least one blood vessel in the region of interest;
b) applying therapy ultrasound to the at least one blood vessel within the region of interest using one or more dosing conditions, the microbubbles interacting with the therapy ultrasound to disrupt the at least one blood vessel;
c) acquiring an image of the region of interest responsive to the applied therapy ultrasound;
d) estimating a change in vascularity of the at least one blood vessel, responsive to the applied therapy ultrasound, using the acquired image to determine whether to adjust at least one of the dosing conditions; and
e) repeating steps (b)-(d) until the at least one blood vessel is disrupted in accordance with a predetermined treatment response,
wherein the applied therapy ultrasound is applied with an intensity to disrupt the at least one blood vessel without damaging a surrounding tissue.

10. The method of claim 9, wherein the microbubbles interact with the therapy ultrasound to disrupt the at least one blood vessel by at least one of forced oscillation, indirect heating or a mechanical shear force.

11. The method of claim 9, step (a) including directing the agent in accordance with an infusion rate.

12. The method of claim 11, further including adjusting the infusion rate in response to the change in vascularity determined in step (d).

13. The method of claim 9, wherein the intensity of the therapy ultrasound is less than or equal to about 5 W/cm².

14. The method of claim 9, wherein the at least one blood vessel is associated with a tumor.

15. A system for treating at least one blood vessel in a region of interest, the system comprising:
a first terminal configured to transmit therapy control parameters for applying therapy ultrasound to the at least one blood vessel within the region of interest, the therapy control parameters including one or more dosing conditions;
a second terminal configured to receive images acquired of the region of interest;
a therapy processor configured to: 1) estimate a change in vascularity of the at least one blood vessel using an image received from the second terminal, responsive to therapy ultrasound applied to the at least one blood vessel and 2) determine whether to adjust at least one of the dosing conditions based on the estimated change in vascularity; and
a controller, coupled to the first terminal and second terminal, configured to transmit the therapy control parameters to control the therapy ultrasound and to control acquisition of the images of the region of interest,
wherein the applied therapy ultrasound has an intensity to disrupt the is blood vessels without damaging a surrounding tissue.

16. The system of claim 15, further comprising:
a therapy ultrasound device coupled to the first terminal to apply the therapy ultrasound to the at least one blood vessel within the region of interest responsive to the controller; and
an imaging device coupled to the second terminal configured to acquire the images of the region of interest responsive to the controller.

17. The system of claim 16, wherein the therapy ultrasound device and the imaging device are configured to be positioned collinear to each other.

18. The system of claim 16, wherein the therapy ultrasound device and the imaging device are configured to be spaced apart from each other.

19. The system of claim 15, further comprising an agent injection device, coupled to the controller, configured to direct an agent including microbubbles to the at least one blood vessel in the region of interest,
wherein the controller is configured to control an infusion rate of the agent responsive to the estimated change in vascularity determined by the therapy processor.

20. The system of claim 15, wherein the therapy ultrasound generates an ultrasound beam, the controller configured to control the ultrasound beam to be minimally focused.

21. The system of claim 15, wherein the intensity of the therapy ultrasound is less than or equal to about 5.0 W/cm².

22. A method of treating a tumor with a therapeutic agent, the method comprising:
introducing the therapeutic agent into a bloodstream to be directed to the tumor;
directing an agent including microbubbles to blood vessels associated with the tumor in a region of interest; and
applying therapy ultrasound to the blood vessels within the region of interest such that the microbubbles interact with the therapy ultrasound to disrupt at least one of the blood vessels,
wherein the therapy ultrasound is applied with an intensity to disrupt the at least one of the blood vessels without damaging a surrounding tissue to limit flow to and from the tumor and to retain the therapeutic agent within the tumor.

23. The method of claim 22, wherein the intensity of the therapy ultrasound is less than or equal to about 5.0 W/cm².

24. The method of claim 22, wherein the applying of the therapy ultrasound includes applying the therapy ultrasound using one or more dosing conditions, the method further including:
acquiring an image of the region of interest responsive to the applied therapy ultrasound; and
estimating a change in vascularity of the at least one blood vessel, responsive to the applied therapy ultrasound, using the acquired image to determine whether to adjust at least one of the dosing conditions.

25. The method of claim 24, the method further comprising:
repeating the applying of the therapy ultrasound, the acquiring of the image and the estimating of the change in vascularity until the estimated change in vascularity corresponds to a predetermined treatment condition.
